(19) United States
(12) Patent Application Publication
(10) Pub. No.: US 2019/0077802 A1
(43) Pub. Date: Mar. 14, 2019

(54) DIFLUORO-(2-SUBSTITUTED CARBAMOYL-1,6-DIAZA-BICYCLO [3.2.1] OCT-6-YLOXY) ACETIC ACID COMPOUNDS AND THEIR USE IN TREATMENT OF BACTERIAL INFECTIONS

(71) Applicant: WOCKHARDT LIMITED, Aurangabad (IN)

(72) Inventors: Vijaykumar Jagdishwar PATIL, Maharashtra (IN); Sushikumar MAURYA, Maharashtra (IN); Bharat DOND, Maharashtra (IN); Velupillai LOGANATHAN, Maharashtra (IN); Deepak DEKHANE, Maharashtra (IN); Prasad DIXIT, Maharashtra (IN); Prashant JOSHI, Parbhani 1 (IN); Swapna TAKALKAR, Maharashtra (IN); Mahesh Vithalbhai PATEL, Maharashtra (IN)

(73) Assignee: WOCKHARDT LIMITED, Aurangabad (IN)

(21) Appl. No.: 15/765,599

(22) PCT Filed: Oct. 5, 2016
(86) PCT No.: PCT/IB2016/055951

§ 371 Date: Apr. 3, 2018

(30) Foreign Application Priority Data
Oct. 6, 2015 (IN) 3785/MUM/2015

(51) Int. Cl. C07D 471/08; A61P 31/04

(52) U.S. Cl. CPC C07D 471/08; A61K 45/06; A61P 31/04

(57) ABSTRACT

The invention discloses compounds of Formula (I), their preparation, and their use in treating a bacterial infection.

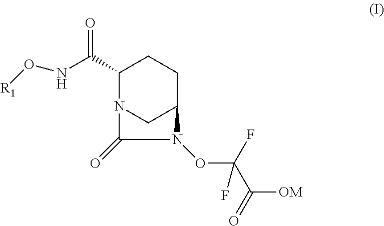

### PRIORITY APPLICATION(S)

This application claims priority to Indian Patent Application No. 3785/MUM/2015 filed on Oct. 6, 2015, the disclosures of which is incorporated herein by reference in its entirety as if fully rewritten herein.

### FIELD OF THE INVENTION

The invention relates to difluoro-(2-substituted carbamoyl-1,6-diazabicyclo[3.2.1]oct-6-yloxy) acetic acid compounds, their preparation and their use in preventing or treating infections.

### BACKGROUND OF THE INVENTION

Emergence of bacterial resistance to known antibacterial agents is becoming a major challenge in treating bacterial infections. One way forward to treat bacterial infections, and especially those caused by resistant bacteria, is to develop newer antibacterial agents that can overcome the bacterial resistant. Coates et al. (Br. J. Pharmacol. 2007; 152(8), 1147-1154.) have reviewed novel approaches to developing new antibiotics. However, the development of new antibacterial agents is a challenging task. For example, Gwynn et al. (Annals of the New York Academy of Sciences, 2010, 1213: 5-19) have reviewed the challenges in discovery of antibacterial agents.

Several antibacterial agents have been described in the prior art (for example, see PCT International Application Nos. PCT/US2010/060923, PCT/EP2010/067647, PCT/US2010/052109, PCT/US2010/048109, PCT/GB2009/050609, PCT/FR01/02418, PCT/EP2009/056178, PCT/US2009/041200, PCT/IB2012/054290, PCT/IB2013/053092, PCT/IB2012/054296, PCT/IB2012/054706, PCT/JP2013/064971, PCT/IB2012/002675, PCT/US2013/034562 and PCT/US2013/034589). However, there remains a need for development of antibacterial agents for preventing and/or treating bacterial infections, including those caused by bacteria that are resistant to known antibacterial agents.

The inventors have now surprisingly discovered difluoro-(2-substituted carbamoyl-1,6-diazabicyclo[3.2.1]oct-6-yloxy) acetic acid compounds having antibacterial activity.

### SUMMARY OF THE INVENTION

Accordingly, there are provided difluoro-(2-substituted carbamoyl-1,6-diazabicyclo[3.2.1]oct-6-yloxy) acetic acid compounds, methods for preparation of these compounds, pharmaceutical compositions comprising these compounds, and methods for preventing or treating bacterial infection in a subject using these compounds.

In one aspect, there are provided compounds of Formula (I):

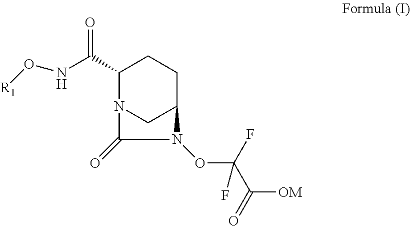

or a stereoisomer or a pharmaceutically acceptable derivative thereof;
wherein:
R₁ is selected from:
(a) hydrogen,
(b) C₁-C₆ alkyl optionally substituted with one or more substituents independently selected from CN, halogen, OR₂, NR₂R₃, COOR₂, CONR₂R₃, SR₂, cycloalkyl, heterocycloalkyl, aryl or heteroaryl,
(c) three to seven membered cycloalkyl optionally substituted with one or more substituents independently selected from CN, halogen, OR₂, NR₂R₃, COOR₂, CONR₂R₃ or SR₂,
(d) three to seven membered heterocycloalkyl optionally substituted with one or more substituents independently selected from CN, halogen, OR₂, NR₂R₃, COOR₂, CONR₂R₃ or SR₂,
(e) six to fourteen membered aryl optionally substituted with one or more substituents independently selected from CN, halogen, OR₂, NR₂R₃, COOR₂, CONR₂R₃ or SR₂, or
(f) five to fourteen membered heteroaryl optionally substituted with one or more substituents independently selected from CN, halogen, OR₂, NR₂R₃, COOR₂, CONR₂R₃ or SR₂;
   R₂ and R₃ are each independently selected from:
(a) hydrogen,
(b) C₁-C₆ alkyl optionally substituted with one or more substituents independently selected from CN, halogen, OH, OCH₃, OC₂H₅, NH₂, NHCH₃, NHC₂H₅, COOH, CONH₂ or SR₂; and
M is hydrogen, cation or C₁-C₆ alkyl.

In another aspect, there are provided pharmaceutical compositions comprising a compound of Formula (I) or a stereoisomer or a pharmaceutically acceptable derivative thereof.

In yet another aspect, there are provided methods for preventing or treating a bacterial infection in a subject, said methods comprising administering to said subject a pharmaceutically effective amount of a compound of Formula (I) or a stereoisomer or a pharmaceutically acceptable derivative thereof.

In another aspect, there are provided methods for preventing or treating a bacterial infection in a subject, said methods comprising administering to said subject a pharmaceutically effective amount of a pharmaceutical composition comprising a compound of Formula (I) or a stereoisomer, or a pharmaceutically acceptable derivative thereof.

In yet another aspect, there are provided pharmaceutical compositions comprising: (a) a compound of Formula (I) or a stereoisomer or a pharmaceutically acceptable derivative thereof, and (b) at least one antibacterial agent or a pharmaceutically acceptable derivative thereof.

In another aspect, there are provided a methods for preventing or treating a bacterial infection in a subject, said methods comprising administering to said subject a pharmaceutically effective amount of a pharmaceutical composition comprising: (a) a compound of Formula (I) or a stereoisomer or a pharmaceutically acceptable derivative thereof, and (b) at least one antibacterial agent or a pharmaceutically acceptable derivative thereof.

In another aspect, there are provided methods for preventing or treating a bacterial infection in a subject, said methods comprising administering to said subject a pharmaceutically effective amount of: (a) a compound of Formula (I) or a stereoisomer or a pharmaceutically acceptable derivative thereof, and (b) at least one antibacterial agent or a pharmaceutically acceptable derivative thereof.

In yet another aspect, there are provided methods for increasing antibacterial effectiveness of an antibacterial agent in a subject, said methods comprising co-administering said antibacterial agent or a pharmaceutically acceptable derivative thereof with a compound of Formula (I) or a stereoisomer or a pharmaceutically acceptable derivative thereof.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The inventors have surprisingly discovered difluoro-(2-substituted carbamoyl-1,6-diazabicyclo[3.2.1]oct-6-yloxy) acetic acid compounds having antibacterial properties. The term “C₁-C₆ alkyl” as used herein refers to branched or unbranched acyclic hydrocarbon radical with 1 to 6 carbon atoms. Typical non-limiting examples of “C₁-C₆ alkyl” include methyl, ethyl, n-propyl, iso-propyl, n-butyl, sec-butyl, iso-butyl, tert-butyl, n-pentyl, iso-pentyl, tert-pentyl, neopentyl, sec-pentyl, 3-pentyl, n-hexyl, 2-methylpentyl, 3-methylpentyl, 2,2-dimethylbutyl, 2,3-dimethylbutyl and the like. The “C₁-C₆ alkyl” may be unsubstituted, or substituted with one or more substituents. Typical, non-limiting examples of such substituents include halogen, alkoxy, CN, SH, COOH, COOC₁-C₆alkyl, CONH₂, OH, NH₂, NHCOCH₃, cycloalkyl, heterocycloalkyl, heteroaryl, aryl and the like.

The term “cycloalkyl” as used herein refers to three to seven member cyclic hydrocarbon radicals. The cycloalkyl group optionally incorporates one or more double or triple bonds, or a combination of double or triple bonds, but which is not aromatic. Typical, non-limiting examples of cycloalkyl groups include cyclopropyl, cyclobutyl, cyclopentyl, cyclohexyl, and cycloheptyl. The cycloalkyl may be unsubstituted, or substituted with one or more substituents. Typical, non-limiting examples of such substituents include C₁-C₆ alkyl, halogen, alkoxy, CN, SH, COOH, COOC₁-C₆alkyl, CONH₂, OH, NH₂, NHCOCH₃, heterocycloalkyl, heteroaryl, aryl, SO₂-alkyl, SO₂-aryl, OSO₂-alkyl, OSO₂-aryl and the like.

The term “aryl” as used herein refers to a monocyclic or polycyclic aromatic hydrocarbon. Typical, non-limiting examples of aryl groups include phenyl, naphthyl, anthracenyl, flourenyl, phenanthrenyl, indenyl and the like. The aryl group may be unsubstituted, or substituted with one or more substituents. Typical, non-limiting examples of such substituents include C₁-C₆ alkyl, halogen, alkoxy, CN, COOH, CONH₂, OH, NH₂, NHCOCH₃, heterocycloalkyl, heteroaryl, aryl, SO₂-alkyl, SO₂-aryl, OSO₂-alkyl, OSO₂-aryl and the like. The term “aryl” includes six to fourteen membered monocyclic or polycyclic aromatic hydrocarbon.

The term “heteroaryl” as used herein refers to a monocyclic or polycyclic aromatic hydrocarbon group wherein one or more carbon atoms have been replaced with heteroatoms selected from nitrogen, oxygen, and sulfur. If the heteroaryl group contains more than one heteroatom, the heteroatoms may be the same or different. Typical, non-limiting example of heteroaryl groups include pyridyl, pyrimidinyl, pyrazinyl, pyridazinyl, triazinyl, furanyl, pyrrolyl, thienyl, oxadiazolyl, thiadiazolyl, tetrazolyl, oxazolyl, thiazolyl, imidazolyl, pyrazolyl, triazonyl, isoxazolyl, oxadiazolyl, oxatriazolyl, isothiazolyl, thiatriazolyl, thiazinyl, oxazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo-pyridazinyl, purinyl, benzofuranyl, isobenzofuranyl, benzothienyl, benzothiophenyl, carbazolyl, benzimidazolyl, benzoxazolyl, benzoisoxazolyl, benzothiazolyl, benzotriazolyl, indolyl, isoindolyl, quinolinyl, isoquinolinyl, acridinyl, naphthothienyl, thianthrenyl, chromenyl, xanthenyl, phenoxathienyl, indolizinyl, indazolyl, phthalazinyl, naphthyridinyl, qinoxalinyl, quinazolinyl, cinnolinyl, pteridinyl, beta-carbolinyl, phenanthridinyl, phenanthrolinyl, phenazinyl, phenothiazinyl, phenoxazinyl and the like. The heteroaryl group may be unsubstituted, or substituted with one or more substituents. Typical, non-limiting examples of such substituents include C₁-C₆ alkyl, halogen, alkoxy, CN, COOH, CONH₂, OH, SH, SCH₃, NH₂, NHCOCH₃, heterocycloalkyl, heteroaryl, aryl, SO₂-alkyl, SO₂-aryl, OSO₂-alkyl, OSO₂-aryl and the like. The term “heteroaryl” includes five to fourteen membered monocyclic or polycyclic aromatic hydrocarbon group containing at least one heteroatom selected from nitrogen, oxygen, and sulfur.

The term “heterocycloalkyl” as used herein refers to three to seven member cycloalkyl group containing one or more heteroatoms selected from nitrogen, oxygen or sulfur. The heterocycloalkyl group optionally incorporates one or more double or triple bonds, or a combination of double bonds and triple bonds, but which is not aromatic. Typical, non-limiting example of heterocycloalkyl groups include aziridinyl, azetidinyl, pyrrolidinyl, 2-oxo-pyrrolidinyl, imidazolidin-2-one-yl, piperidinyl, oxazinyl, thiazinyl, piperazinyl, piperazin-2,3-dione-yl, morpholinyl, thiomorpholinyl, azepanyl, and the like. The heterocycloalkyl may be unsubstituted, or substituted with one or more substituents. Typical, non-limiting examples of such substituents include C₁-C₆ alkyl, halogen, alkoxy, CN, COOH, CONH₂, OH, NH₂, NHCOCH₃, heteroaryl, aryl, SO₂-alkyl, SO₂-aryl, OSO₂-aryl and the like. The term “heterocycloalkyl” includes three to seven membered cycloalkyl containing at least one heteroatom selected from nitrogen, oxygen, and sulfur.

The term “halogen” or halo as used herein refers to chlorine, bromine, fluorine or iodine.

The term “Bn” as used herein refers to benzyl group.

The term “Boc” as used herein refers to tert-butyloxycarbonyl group

The term “stereoisomers” as used herein refers to compounds that have identical chemical constitution, but differ with regard to the arrangement of their atoms or groups in space. The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, are embraced within the scope of the invention. In general, a reference to a compound is intended to cover its stereoisomers and mixture of various stereoisomers.

The term “optionally substituted” as used herein means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound.

The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the parent compound. For example, the term “antibacterial agent or a pharmaceutically acceptable derivative thereof” includes all derivatives of the antibacterial agent (such as salts, pro-drugs, metabolites, esters, ethers, hydrates, polymorphs, solvates, complexes, and adducts) which, upon administration to a subject, are capable of providing (directly or indirectly) the antibacterial agent.

The term “pharmaceutically acceptable salt” as used herein refers to one or more salts of a given compound which possesses the desired pharmacological activity of the free compound and which are neither biologically nor otherwise undesirable. In general, the “pharmaceutically acceptable salts” refer to salts that are suitable for use in contact with the tissues of human and animals without undue toxicity, irrigation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutical acceptable salts in details.

In general, the compounds according to the invention contain basic (e.g. nitrogen atoms) as well as acid moieties (e.g. compounds of Formula (I) wherein M is hydrogen). A person of skills in the art would appreciate that such compounds, therefore, can form acidic salts (formed with inorganic and/or organic acids), as well as basic salts (formed with inorganic and/or organic bases). Such salts can be prepared using procedures described in the art. For example, the basic moiety can be converted to its salt by treating a compound with a suitable amount of acid. Typical, non-limiting examples of such suitable acids include hydrochloric acid, trifluoroacetic acid, methanesulfonic acid or the like. Alternatively, the acid moiety may be converted into its salt by treating with a suitable base. Typical non-limiting examples of such bases include sodium carbonate, sodium bicarbonate, sodium ethylhexanoate, potassium carbonate, potassium bicarbonate, potassium ethyl hexanoate or the like. In case of compounds containing more than one functional group capable of being converted into salt, each such functional group may be converted to salt independently. For example, in case of compounds containing two basic nitrogen atoms, one of the basic nitrogen can form salt with one acid while the other basic nitrogen can form salt with another acid. Some compounds according to the invention contain both acidic as well as basic moieties, and thus can form inner salts or corresponding zwitterions. In general, all pharmaceutically acceptable salt forms of compound of Formula (I) according to invention including acid addition salts, base addition salts, zwitterions or the like are contemplated to be within the scope of the present invention and are generically referred to as pharmaceutically acceptable salts.

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of other floras, which are not desirable. The term “infection” includes infection caused by bacteria.

The term “treat”, “treating” or “treatment” as used herein refers to administration of a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions, or one or more of pharmaceutically active ingredients discussed herein, with or without additional pharmaceutically active or inert ingredients, in order to: (i) reduce or eliminate either a bacterial infection, or one or more symptoms of a bacterial infection, or (ii) retard progression of a bacterial infection, or one or more symptoms of a bacterial infection, or (iii) reduce severity of a bacterial infection, or one or more symptoms of a bacterial infection, or (iv) suppress clinical manifestation of a bacterial infection, or (v) suppress manifestation of adverse symptoms of a bacterial infection.

The terms “pharmaceutically effective amount” or “therapeutically effective amount” or “effective amount” as used herein refer to an amount, which has a therapeutic effect or is the amount required to produce a therapeutic effect in a subject. For example, a “therapeutically effective amount” or “pharmaceutically effective amount” or “effective amount” of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” refers to and includes delivery of a composition, or one or more pharmaceutically active ingredients to a subject, including for example, by any appropriate method, which serves to deliver the composition or its active ingredients or other pharmaceutically active ingredients to the site of infection. The method of administration may vary depending on various factors, such as for example, the components of the pharmaceutical composition or type/nature of the pharmaceutically active or inert ingredients, site of the potential or actual infection, the microorganism involved, severity of the infection, age and physical condition of the subject and a like. Some non-limiting examples of ways to administer a composition or a pharmaceutically active ingredient to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop and mouthwash. In case of a pharmaceutical composition comprising more than one ingredients (active or inert), one of the ways of administering such composition is by admixing the ingredients (e.g. in the form of a suitable unit dosage form such as tablet, capsule, solution, powder or a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g. bacteria). The term “growth” also includes maintenance of on-going metabolic processes of the microorganism, including the processes that keep the microorganism alive.

The term, “effectiveness” as used herein refers to ability of a treatment, or a composition, or one or more pharmaceutically active ingredients to produce a desired biological effect in a subject. For example, the term “antibacterial effectiveness” of a composition or of an antibacterial agent refers to the ability of the composition or the antibacterial agent to prevent or treat bacterial infection in a subject.

The term “synergistic” or “synergy” as used herein refers to the interaction of two or more agents so that their combined effect is greater than their individual effects.

The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

The term “beta-lactamase” or “beta-lactamase enzyme” as used herein refers to any enzyme or protein or any other substance that breaks down a beta-lactam ring. The term “beta-lactamase” includes enzymes that are produced by bacteria and have the ability to hydrolyze the beta-lactam ring in a beta-lactam compound, either partially or completely.

The term “beta-lactamase inhibitor” as used herein refers to a compound capable of inhibiting activity of one or more beta-lactamase enzymes, either partially or completely.

The term “pharmaceutically inert ingredient” or “carrier” or “excipient” refers to and includes compounds or materials used to facilitate administration of a compound, for example, to increase the solubility of the compound. Typical, non-limiting examples of solid carriers include starch, lactose, dicalcium phosphate, sucrose, and kaolin. Typical, non-limiting examples of liquid carriers include sterile water, saline, buffers, non-ionic surfactants, and edible oils. In addition, various adjuvants commonly used in the art may also be included. These and other such compounds are described in literature, e.g., in the Merck Index (Merck & Company, Rahway, N.J.). Considerations for inclusion of various components in pharmaceutical compositions are described, e.g., in Gilman et al. (Goodman and Gilman's: The Pharmacological Basis of Therapeutics, 8th Ed., Pergamon Press., 1990), which is incorporated herein by reference in its entirety.

The term “subject” as used herein refers to vertebrate or invertebrate, including a mammal. The term “subject” includes human, animal, a bird, a fish, or an amphibian. Typical, non-limiting examples of a “subject” include humans, cats, dogs, horses, sheep, bovine cows, pigs, lambs, rats, mice and guinea pigs.

The term “cation” as used herein refers to all organic and inorganic positively charged ions. The term “organic cation” refers to all positively charged organic ions. Typical, non-limiting examples of organic cations include unsubstituted ammonium cations, alkyl substituted ammonium cations, cycloalkyl substituted ammonium cations, primary, secondary and tertiary amines, alkyl amines, cycloalkyl amines, aryl amines, N,N′-dibenzylethylenediamine and the like. The term “inorganic cation” refers to a positively charged metal ion. Typical, non-limiting examples of inorganic cations include Group I and Group II metal cations such as, for example, lithium, sodium, potassium, rubidium, cesium, beryllium, magnesium, calcium and the like.

In one aspect, there are provided compounds of Formula (I):

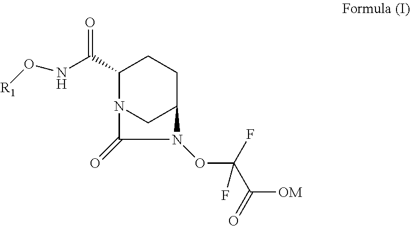

or a stereoisomer or a pharmaceutically acceptable derivative thereof;
wherein:
R₁ is selected from:
(a) hydrogen,
(b) C₁-C₆ alkyl optionally substituted with one or more substituents independently selected from CN, halogen, OR₂, NR₂R₃, COOR₂, CONR₂R₃, SR₂, cycloalkyl, heterocycloalkyl, aryl or heteroaryl,
(c) three to seven membered cycloalkyl optionally substituted with one or more substituents independently selected from CN, halogen, OR₂, NR₂R₃, COOR₂, CONR₂R₃ or SR₂,
(d) three to seven membered heterocycloalkyl optionally substituted with one or more substituents independently selected from CN, halogen, OR₂, NR₂R₃, COOR₂, CONR₂R₃ or SR₂,
(e) six to fourteen membered aryl optionally substituted with one or more substituents independently selected from CN, halogen, OR₂, NR₂R₃, COOR₂, CONR₂R₃, SR₂, or
(f) five to fourteen membered heteroaryl optionally substituted with one or more substituents independently selected from CN, halogen, OR₂, NR₂R₃, COOR₂, CONR₂R₃ or SR₂;
   R₂ and R₃ are each independently selected from:
(a) hydrogen,
(b) C₁-C₆ alkyl optionally substituted with one or more substituents independently selected from CN, halogen, OH, OCH₃, OC₂H₅, NH₂, NHCH₃, NHC₂H₅, COOH, CONH₂ or SR₂; and
M is hydrogen, cation or C₁-C₆ alkyl.

Typical, non-limiting examples of compounds according to the invention include:
Difluoro {[(2S,5R)-[7-oxo-2-{[(2S)-pyrrolidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2S)-pyrrolidin-2-ylmethoxy]carbamoyl}-1,6-diaza-bicyclo[3.2.1]oct-6-yloxy]acetic acid;
Difluoro {[(2S,5R)-[7-oxo-2-{[(2R)-pyrrolidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2R)-pyrrolidin-2-ylmethoxy]carbamoyl}-1,6-diaza-bicyclo[3.2.1]oct-6-yloxy]acetic acid;
Difluoro {[(2S,5R)-[7-oxo-2-{[(3S)-pyrrolidin-3-yloxy]carbamoyl}-1,6-diaza-bicyclo [3.2.1]oct-6-yloxy]acetic acid ethyl ester;
Difluoro {[(2S,5R)-[7-oxo-2-{[(3S)-pyrrolidin-3-yloxy]carbamoyl}-1,6-diaza-bicyclo [3.2.1]oct-6-yloxy]acetic acid;
Difluoro {[(2S,5R)-[7-oxo-2-{[(3R)-pyrrolidin-3-yloxy]carbamoyl}-1,6-diaza-bicyclo [3.2.1]oct-6-yloxy]acetic acid ethyl ester;
Difluoro {[(2S,5R)-[7-oxo-2-{[(3R)-pyrrolidin-3-yloxy]carbamoyl}-1,6-diaza-bicyclo[3.2.1]oct-6-yloxy]acetic acid;
Difluoro {[(2S,5R)-[7-oxo-2-{[(2R)-piperidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2R)-piperidin-2-ylmethoxy]carbamoyl}-1,6-diaza-bicyclo[3.2.1]oct-6-yloxy]acetic acid;
Difluoro {[(2S,5R)-[7-oxo-2-{[(2S)-piperidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2S)-piperidin-2-ylmethoxy]carbamoyl}-1,6-diaza-bicyclo[3.2.1]oct-6-yloxy]acetic acid;
Difluoro {[(2S,5R)-[7-oxo-2-{[(2R)-azepan-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2R)-azepan-2-ylmethoxy]carbamoyl}-1,6-diaza-bicyclo[3.2.1]oct-6-yloxy]acetic acid;
Difluoro {[(2S,5R)-[7-oxo-2-{[(2S)-azepan-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2S)-azepan-2-ylmethoxy]carbamoyl}-1,6-diaza-bicyclo[3.2.1]oct-6-yloxy]acetic acid;
({(2S,5R)-2-[(2-aminoethoxy)carbamoyl]-7-oxo-1,6-diazabicyclo[3.2.1]oct-6-yl}oxy)(difluoro)acetic acid ethyl ester;
({(2S,5R)-2-[(2-aminoethoxy)carbamoyl]-7-oxo-1,6-diazabicyclo[3.2.1]oct-6-yl}oxy)(difluoro)acetic acid;
Difluoro {[(2S,5R)-[7-oxo-2-{[(2R)-azetidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2R)-azetidin-2-ylmethoxy]carbamoyl}-1,6-diaza-bicyclo[3.2.1]oct-6-yloxy]acetic acid;
Difluoro {[(2S,5R)-[7-oxo-2-{[(2S)-azetidin-2-ylmethoxy]carbamoyl}-1,6-diaza-bicyclo[3.2.1]oct-6-yloxy]acetic acid ethyl ester;
Difluoro {[(2S,5R)-[7-oxo-2-{[(2S)-azetidin-2-ylmethoxy]carbamoyl}-1,6-diaza-bicyclo[3.2.1]oct-6-yloxy]acetic acid;

or a stereoisomer or a pharmaceutically acceptable derivative thereof.

In some other embodiments, typical, non-limiting examples of compounds according to the invention include:
Sodium salt of difluoro {[(2S,5R)-[7-oxo-2-{[(2S)-pyrrolidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2R)-pyrrolidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(3S)-pyrrolidin-3-yloxy]carbamoyl}-1,6-diaza-bicyclo[3.2.1]oct-6-yloxy]acetic acid;
Sodium salt of difluoro {[(2S,5R)-[7-oxo-2-{[(3R)-pyrrolidin-3-yloxy]carbamoyl}-1,6-diaza-bicyclo[3.2.1]oct-6-yloxy]acetic acid;
Sodium salt of difluoro {[(2S,5R)-[7-oxo-2-{[(2R)-piperidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-piperidin-2-ylmethoxy]carbamoyl}-1,6-diaza-bicyclo[3.2.1]oct-6-yloxy]acetic acid;
Sodium salt of difluoro{[(2S,5R)-[7-oxo-2-{[(2R)-azepan-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-azepan-2-ylmethoxy]carbamoyl}-1,6-diaza-bicyclo[3.2.1]oct-6-yloxy]acetic acid;
Sodium salt of ({(2S,5R)-2-[(2-aminoethoxy)carbamoyl]-7-oxo-1,6-diazabicyclo[3.2.1]oct-6-yl}oxy)(difluoro)acetic acid;
Sodium salt of difluoro {[(2S,5R)-[7-oxo-2-{[(2R)-azetidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-azetidin-2-ylmethoxy]carbamoyl}-1,6-diaza-bicyclo[3.2.1]oct-6-yloxy]acetic acid;

or a stereoisomer thereof.

In general, the compounds of the invention can be prepared according to the general procedures given in Scheme 1. A person of skills in the art would appreciate that the described method can be varied or optimized further to provide the desired and related compounds. In the following procedures all variables are as defined above.

In one general aspect, the compounds according to invention are prepared according to general procedure given in Scheme 1. The compound of Formula (II) (prepared according to process disclosed in International Application Number PCT/IB2013/053092) is debenzylated by carrying out hydrogenolysis in presence of hydrogen, transition metal catalyst and a suitable solvent at a temperature ranging from about 10° C. to about 60° C. for about 1 hour to about 14 hours to obtain a compound of Formula (III). In the compound of Formula (II) R₁′ is the protected R₁. Typical, non-limiting examples of hydrogen source include hydrogen gas, ammonium formate, cyclohexene, lithium-liquid ammonia, ammonia-tert-butanol, sodium-liquid ammonia-tert-butanol, triethyl silyl hydride and the like. Typical, non-limiting examples of transition metal catalyst include 5% palladium on carbon, 10% palladium on carbon, 20% palladium hydroxide on carbon, Raney-Nickel and the like. Typical, non-limiting examples of suitable solvent include methanol, ethanol, dichloromethane, N,N dimethylformamide, ethyl acetate, tetrahydrofuran, and the like, or a mixture thereof. In some embodiments, compound of Formula (II) is treated with 10% palladium on carbon in presence of hydrogen gas at 20-30 psi and at temperature of about 25° C. for about 2 hours to provide a debenzylated compound. In some embodiments, compound of Formula (II) is converted to a compound of Formula (III) in presence of mixture of tetrahydrofuran and ethyl acetate as a solvent.

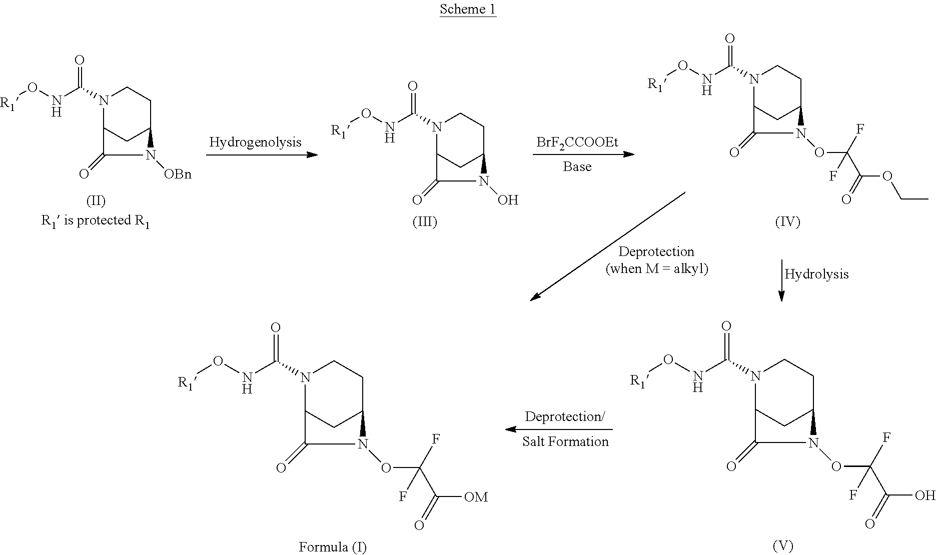

The compound of Formula (III) is further converted to a compound of Formula (IV). In some embodiments, the compound of Formula (III) is treated with ethyl bromodifluoroacetate in presence of a suitable base and suitable solvent to obtain a compound of Formula (IV). Typical, non-limiting examples of suitable base include potassium carbonate, cesium carbonate, sodium hydride, potassium-tert-butoxide, sodium bicarbonate and the like. Typical, non-limiting examples of suitable solvent include dimethylformamide, tetrahydrofuran, dioxane and the like. In some embodiments, compound of Formula (IV) is directly converted to a compound of Formula (I), wherein M is C₁-C₆ alkyl. In some embodiments, compound of Formula (IV) is deprotected in presence of a suitable deprotecting agent to obtain a compound of Formula (I), wherein M is C₁-C₆ alkyl.

The compound of Formula (IV) is hydrolyzed to obtain a compound of Formula (V). In some embodiments, compound of Formula (IV) is treated with a suitable hydrolyzing agent in presence of a suitable solvent at a temperature of about −10° C. to about 0° C. to obtain a compound of Formula (V). Typical, non-limiting examples of a suitable solvent include tetrahydrofuran, water, dioxane and the like. Typical, non-limiting examples of suitable hydrolyzing agents include lithium hydroxide, sodium hydroxide and the like. In some embodiments, a compound of Formula (IV) is treated with lithium hydroxide in presence of a mixture of tetrahydrofuran and water as a solvent at a temperature of about −5° C. to obtain a compound of Formula (V).

The compound of Formula (V) is deprotected to obtain a compound of Formula (I). The R₁′ of a compound of Formula (V) is converted to R₁ of a compound of Formula (I). In some embodiments, a compound of Formula (V) is treated with a suitable deprotecting agent in presence of a suitable solvent and at a temperature of about −20° C. to about 40° C. for about 0.5 hour to about 14 hours to obtain a compound of Formula (I). Typical, non-limiting examples of a suitable deprotecting agent include trifluoroacetic acid, hydrochloric acid and the like. Typical, non-limiting examples of a suitable solvent include dichloromethane, chloroform, acetonitrile, dioxane and the like. In some embodiments, compound of Formula (V) is treated with trifluoroacetic acid in presence of dichloromethane at temperature of about −10° C. to about −5° C. for about 4 hours to provide a compound of Formula (I).

In some embodiments, compounds according to invention are isolated as pharmaceutically acceptable salts. In some embodiments, compounds according to invention are isolated as sodium salts, wherein a compound of Formula (I) is dissolved in suitable solvent and passed through cation exchange resin. In some other embodiments, compound of Formula (I) is dissolved in 10% tetrahydrofuran: water mixture and then passed through the column packed with cation exchange resin such as Dowex 50WX8 200 Sodium resin, Indion 225 Sodium resin and the like. In some embodiments, compound of Formula (I) is dissolved in suitable solvent such as acetone, tetrahydrofuran, ethanol, isopropanol, acetonitrile, and the like, and treated with sodium ethylhexanoate or potassium ethylhexanoate to provide corresponding sodium or potassium salt of compound of Formula (I).

In some embodiments, there are provided pharmaceutical compositions comprising a compound of Formula (I) or a stereoisomer or a pharmaceutically acceptable derivative thereof.

In some other embodiments, there are provided pharmaceutical compositions comprising: (a) a compound of Formula (I) or a stereoisomer or a pharmaceutically acceptable derivative thereof, and (b) at least one beta-lactamase inhibitor or a pharmaceutically acceptable derivative thereof.

In some other embodiments, there are provided pharmaceutical compositions comprising: (a) a compound of Formula (I) or a stereoisomer or a pharmaceutically acceptable derivative thereof, and (b) at least one beta-lactamase inhibitor selected from sulbactam, tazobactam, clavulanic acid, avibactam or a pharmaceutically acceptable derivative thereof.

In some other embodiments, there are provided pharmaceutical compositions comprising: (a) a compound of Formula (I) or a stereoisomer or a pharmaceutically acceptable derivative thereof, and (b) at least one antibacterial agent or a pharmaceutically acceptable derivative thereof.

In some other embodiments, there are provided pharmaceutical compositions comprising: (a) a compound of Formula (I) or a stereoisomer or a pharmaceutically acceptable derivative thereof, and (b) at least one antibacterial agent selected from cefepime, cefpirome, ceftaroline, ceftazidime, ceftalozane or a pharmaceutically acceptable derivative thereof.

In some other embodiments, there are provided pharmaceutical compositions comprising: (a) a compound of Formula (I) or a stereoisomer or a pharmaceutically acceptable derivative thereof, (b) at least one beta-lacatamase inhibitor or a pharmaceutically acceptable derivative thereof, and (c) at least one antibacterial agent, or a pharmaceutically acceptable derivative thereof.

In some other embodiments, there are provided methods for preventing or treating a bacterial infection in a subject, said methods comprising administering to said subject a pharmaceutical composition comprising a compound of Formula (I) or a stereoisomer or a pharmaceutically acceptable derivative thereof.

In some other embodiments, there are provided methods for preventing or treating a bacterial infection in a subject, said methods comprising administering to said subject a pharmaceutical composition comprising: (a) a compound of Formula (I), or a stereoisomer or a pharmaceutically acceptable derivative thereof and (b) at least one beta-lactamase inhibitor or pharmaceutically acceptable derivative thereof.

In some other embodiments, there are provided methods for preventing or treating a bacterial infection in a subject, said methods comprising administering to said subject a pharmaceutical composition comprising: (a) a compound of Formula (I), or a stereoisomer or a pharmaceutically acceptable derivative thereof and (b) at least one beta-lactamase inhibitor selected from sulbactam, tazobactam, clavulanic acid, avibactam, or pharmaceutically acceptable derivative thereof.

In some other embodiments, there are provided methods for preventing or treating a bacterial infection in a subject, said methods comprising administering to said subject a pharmaceutical composition comprising: (a) a compound of Formula (I) or a stereoisomer or a pharmaceutically acceptable derivative thereof, and (b) at least one antibacterial agent or a pharmaceutically acceptable derivative thereof.

In some other embodiments, there are provided methods for preventing or treating a bacterial infection in a subject, said methods comprising administering to said subject a pharmaceutical composition comprising: (a) a compound of Formula (I) or a stereoisomer or a pharmaceutically acceptable derivative thereof, and (b) at least one antibacterial agent selected from selected from cefepime, cefpirome, ceftaroline, ceftazidime, ceftalozane or a pharmaceutically acceptable derivative thereof.

In some other embodiments, there are provided methods for preventing or treating a bacterial infection in a subject, said methods comprising administering to said subject a pharmaceutical composition comprising: (a) a compound of Formula (I) or a stereoisomer or a pharmaceutically acceptable derivative thereof, (b) at least one beta-lactamase inhibitor or pharmaceutically acceptable derivative thereof and (c) at least one antibacterial agent or a pharmaceutically acceptable derivative thereof.

In some other embodiments, there are provided methods for preventing or treating a bacterial infection in a subject, said method comprising administering to said subject a compound of Formula (I) or a stereoisomer or a pharmaceutically acceptable derivative thereof.

In some other embodiments, there are provided methods for preventing or treating a bacterial infection in a subject, said methods comprising administering to said subject: (a) a compound of Formula (I) or a stereoisomer or a pharmaceutically acceptable derivative thereof, (b) at least one beta-lactamase inhibitor or pharmaceutically acceptable derivative thereof.

In some other embodiments, there are provided methods for preventing or treating a bacterial infection in a subject, said methods comprising administering to said subject: (a) a compound of Formula (I) or a stereoisomer or a pharmaceutically acceptable derivative thereof, (b) at least one beta-lactamase inhibitor selected from sulbactam, tazobactam, clavulanic acid, avibactam, or pharmaceutically acceptable derivative thereof.

In some other embodiments, there are provided methods for preventing or treating a bacterial infection in a subject, said methods comprising administering to said subject: (a) a compound of Formula (I) or a stereoisomer or a pharmaceutically acceptable derivative thereof, (b) at least one antibacterial agent or pharmaceutically acceptable derivative thereof.

In some other embodiments, there are provided methods for preventing or treating a bacterial infection in a subject, said methods comprising administering to said subject: (a) a compound of Formula (I) or a stereoisomer or a pharmaceutically acceptable derivative thereof, (b) at least one antibacterial agent selected from selected from cefepime, cefpirome, ceftaroline, ceftazidime, ceftalozane or pharmaceutically acceptable derivative thereof.

In some other embodiments, there are provided methods for preventing or treating a bacterial infection in a subject, said methods comprising administering to said subject: (a) a compound of Formula (I) or a stereoisomer or a pharmaceutically acceptable derivative thereof, (b) at least one beta-lactamase inhibitor or pharmaceutically acceptable derivative thereof, and (c) at least one antibacterial agent or pharmaceutically acceptable derivative thereof.

In some embodiments, the compositions and methods according to the invention use compounds of Formula (I), or a stereoisomer or a pharmaceutically acceptable derivative thereof in combination with at least one antibacterial agent or a pharmaceutically acceptable derivative thereof. A wide variety of antibacterial agents can be used. Typical, non-limiting examples of antibacterial agents include one or more of antibacterial compounds generally classified as aminoglycosides, ansamycins, carbacephems, cephalosporins, cephamycins, lincosamides, lipopeptides, macrolides, monobactams, nitrofurans, penicillins, polypeptides, quinolones, sulfonamides, tetracyclines, oxazolidinone and the like. Typical, non-limiting examples of aminoglycoside antibacterial agents include amikacin, gentamicin, kanamycin, neomycin, netilmicin, tobramycin, paromomycin, arbekacin, streptomycin, apramycin and the like. Typical, non-limiting examples of ansamycin antibacterial agents include geldanamycin, herbimycin and the like. Typical, non-limiting examples of carbacephem antibacterial agents include loracarbef and the like. Typical, non-limiting examples of carbapenem antibacterial agents include ertapenem, doripenem, imipenem, meropenem and the like.

Typical, non-limiting examples of cephalosporin and cephamycin antibacterial agents include cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefmetazole, carbacephem, cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, cxacephem, cefepime, cefozopran, cefpirome, cefquinome, ceftobiprole, ceftiofur, cefquinome, cefovecin, ceftolozane, ceftaroline, ceftobiprole and the like

Typical, non-limiting examples of lincosamide antibacterial agents include clindamycin, lincomycin and the like. Typical, non-limiting examples of macrolide antibacterial agents include azithromycin, clarithromycin, dirithromycin, erythromycin, roxithromycin, troleandomycin, telithromycin, spectinomycin, solithromycin and the like. Typical, non-limiting examples of monobactam antibacterial agents include aztreonam and the like. Typical, non-limiting examples of nitrofuran antibacterial agents include furazolidone, nitrofurantoin and the like. Typical, non-limiting examples of penicillin antibacterial agents include amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin and the like. Typical, non-limiting examples of polypeptide antibacterial agents include bacitracin, colistin, polymyxin B and the like.

Typical, non-limiting examples of quinolone antibacterial agents include ciprofloxacin, enoxacin, gatifloxacin, levofloxacin, lomefloxacin, moxifloxacin, nalidixic acid, levonadifloxacin, norfloxacin, ofloxacin, trovafloxacin, grepafloxacin, sparfloxacin, temafloxacin and the like. Typical, non-limiting examples of sulfonamide antibacterial agents include mafenide, sulfonamidochrysoidine, sulfacetamide, sulfadiazine, sulfamethizole, sulfamethoxazole, sulfasalazine, sulfisoxazole, trimethoprim and the like. Typical, non-limiting examples of tetracycline antibacterial agents include demeclocycline, doxycycline, minocycline, oxytetracycline, tetracycline, tigecycline and the like. Typical, non-limiting examples of oxazolidinone antibacterial agents include tedizolid, linezolid, ranbezolid, torezolid, radezolid and the like.

The pharmaceutical compositions according to the invention may include one or more pharmaceutically acceptable carriers or excipients or the like, Typical, non-limiting examples of such carriers or excipient include mannitol, lactose, starch, magnesium stearate, sodium saccharine, talcum, cellulose, sodium crosscarmellose, glucose, gelatin, sucrose, magnesium carbonate, wetting agents, emulsifying agents, solubilizing agents, pH buffering agents, lubricants, stabilizing agents, binding agents etc.

In some embodiments, pharmaceutical compositions according to the present invention are administered orally or parenterally.

The pharmaceutical compositions according to this invention can exist in various forms. In some embodiments, the pharmaceutical composition is in the form of a powder or a solution. In some other embodiments, the pharmaceutical compositions according to the invention are in the form of a powder that can be reconstituted by addition of a compatible reconstitution diluent prior to parenteral administration. Non-limiting example of such a compatible reconstitution diluent includes water.

In some other embodiments, the pharmaceutical compositions according to the invention are in the form of a frozen composition that can be diluted with a compatible diluent prior to parenteral administration.

In some other embodiments, the pharmaceutical compositions according to the invention are in the form ready to use for oral or parenteral administration.

In the methods according to the invention, the pharmaceutical composition and/or other pharmaceutically active ingredients disclosed herein may be administered by any appropriate method, which serves to deliver the composition or its constituents or the active ingredients to the desired site. The method of administration can vary depending on various factors, such as for example, the components of the pharmaceutical composition and nature of the active ingredients, the site of the potential or actual infection, the microorganism (e.g. bacteria) involved, severity of infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash.

The compositions according to the invention can be formulated into various dosage forms wherein the active ingredients and/or excipients may be present either together (e.g. as an admixture) or as separate components. When the various ingredients in the composition are formulated as a mixture, such composition can be delivered by administering such a mixture to a subject using any suitable route of administration. Alternatively, pharmaceutical compositions according to the invention may also be formulated into a dosage form wherein one or more ingredients (active or inactive ingredients) are present as separate components. The composition or dosage form wherein the ingredients do not come as a mixture, but come as separate components, such composition/dosage form may be administered in several ways. In one possible way, the ingredients may be mixed in the desired proportions and the mixture is then administered as required. Alternatively, the components or the ingredients (active or inert) may be separately administered (simultaneously or one after the other) in appropriate proportion so as to achieve the same or equivalent therapeutic level or effect as would have been achieved by administration of the equivalent mixture.

In some embodiments, pharmaceutical compositions according to the invention are formulated into a dosage form such that the compound of Formula (I) or a stereoisomer or a pharmaceutically acceptable derivative thereof, and the antibacterial agent or a pharmaceutically acceptable derivative thereof, are present in the composition as admixture or as separate components. In some other embodiments, pharmaceutical compositions according to the invention are formulated into a dosage form such that the compound of Formula (I) or a stereoisomer or a pharmaceutically acceptable derivative thereof, and the antibacterial agent or a pharmaceutically acceptable derivative thereof, are present in the composition as separate components.

Similarly, in the methods according to the invention, the active ingredients disclosed herein may be administered to a subject in several ways depending on the requirements. In some embodiments, the active ingredients are admixed in appropriate amounts and then the admixture is administered to a subject. In some other embodiments, the active ingredients are administered separately. Since the invention contemplates that the active ingredients agents may be administered separately, the invention further provides for combining separate pharmaceutical compositions in kit form. The kit may comprise one or more separate pharmaceutical compositions, each comprising one or more active ingredients. Each of such separate compositions may be present in a separate container such as a bottle, vial, syringes, boxes, bags, and the like. Typically, the kit comprises directions for the administration of the separate components. The kit form is particularly advantageous when the separate components are preferably administered in different dosage forms (e.g., oral and parenteral) ore are administered at different dosage intervals. When the active ingredients are administered separately, they may be administered simultaneously or sequentially.

The pharmaceutical composition or the active ingredients according to the present invention may be formulated into a variety of dosage forms. Typical, non-limiting examples of dosage forms include solid, semi-solid, liquid and aerosol dosage forms; such as tablets, capsules, powders, solutions, suspensions, suppositories, aerosols, granules, emulsions, syrups, elixirs and a like.

In general, the pharmaceutical compositions and method disclosed herein are useful in preventing or treating bacterial infections. In some embodiments, the compound of Formula (I) or a stereoisomer or a pharmaceutically acceptable derivative thereof is used in a medicament useful in the treatment of a bacterial infection. In some other embodiments, pharmaceutical composition comprising a compound of Formula (I) or a stereoisomer or a pharmaceutically acceptable derivative thereof is used as a medicament useful in the treatment of a bacterial infection.

Advantageously, the compositions and methods disclosed herein are also effective in preventing or treating infections caused by bacteria that are considered be less or not susceptible to one or more of known antibacterial agents or their known compositions. Some non-limiting examples of such bacteria known to have developed resistance to various antibacterial agents include Acinetobacter, E. coli, Pseudomonas aeruginosa, Staphylococcus aureus, Enterobacter, Klebsiella, Citrobacter and a like. Other non-limiting examples of infections that may be prevented or treated using the compositions and/or methods of the invention include: skin and soft tissue infections, febrile neutropenia, urinary tract infection, intraabdominal infections, respiratory tract infections, pneumonia (nosocomial), bacteremia meningitis, surgical, infections etc.

Surprisingly, the compounds, compositions and methods according to the invention are also effective in preventing or treating bacterial infections that are caused by bacteria producing one or more beta-lactamase enzymes. In some embodiments, there are provided methods of inhibiting beta-lactamase enzymes, wherein said methods comprise administering a pharmaceutically effective amount of a compound of Formula (I), or a stereoisomer or a pharmaceutically acceptable derivative thereof. In some other embodiments, there are provided methods of inhibiting beta-lactamase enzymes, wherein said methods comprise administering a pharmaceutically effective amount of a pharmaceutical composition comprising a compound of Formula (I), or a stereoisomer or a pharmaceutically acceptable derivative thereof.

In general, the compounds of Formula (I), or a stereoisomer or pharmaceutically acceptable salt thereof according to invention are also useful in increasing antibacterial effectiveness of antibacterial agent in a subject. The antibacterial effectiveness of one or more antibacterial agents may increased, for example, by co-administering said antibacterial agent or a pharmaceutically acceptable derivative thereof with a pharmaceutically effective amount of a compound of Formula (I), or a stereoisomer or a pharmaceutically acceptable salt thereof according to the invention. In some embodiments, there is provided a method for increasing antibacterial effectiveness of the antibacterial agent in a subject, said method comprising co-administering said antibacterial agent or a pharmaceutically acceptable derivative thereof with a of a compound of Formula (I), or a stereoisomer or a pharmaceutically acceptable derivative thereof.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Synthesis of difluoro{[(2S,5R)-[7-oxo-2-{[(2S)-pyrrolidin-2-ylmethoxy]carbamoyl}-1,6-diaza-bicyclo[3.2.1]oct-6-yloxy]acetic acid

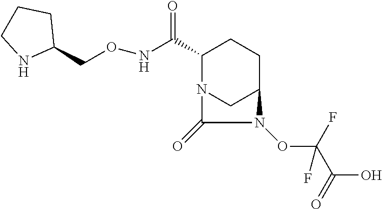 [SMILES: O=C(NOC[C@@H]1CCCN1)[C@@H]1CC[C@@H]2CN1C(=O)N2OC(F)(F)C(=O)O]

### Step 1: Synthesis of (2S,5R)-2-[(6-hydroxy-7-oxo-1,6-diaza-bicyclo[3.2.1]octane-2-carbonyl)-methoxycarbamoyl]-(2S)-pyrrolidine-1-carboxylic acid tert-butyl ester

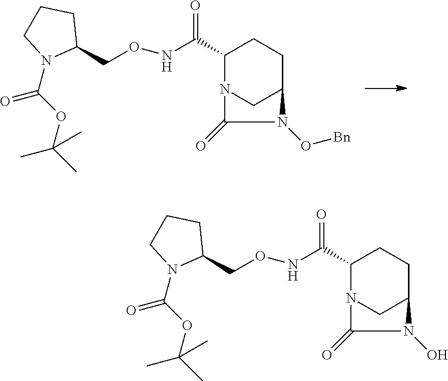 [SMILES: CC(C)(C)OC(=O)N1CCC[C@H]1CONC(=O)[C@@H]1CC[C@@H]2CN1C(=O)N2OCc1ccccc1 ; CC(C)(C)OC(=O)N1CCC[C@H]1CONC(=O)[C@@H]1CC[C@@H]2CN1C(=O)N2O]

A solution of (2S,5R)-2-[(6-benzyloxy-7-oxo-1,6-diaza-bicyclo[3.2.1]octane-2-carbonyl)-methoxycarbamoyl]-(2S)-pyrrolidine-1-carboxylic acid tert-butyl ester (prepared according to process disclosed in International Application Number PCT/IB2013/053092) (6.0 g, 0.02 mol) in a mixture of dichloromethane and dimethylformamide (60 ml, 1:1), containing 10% palladium over carbon (1.5 g, 50% wet) was hydrogenated under 4.5 kg of hydrogen pressure. After 4 hours, the catalyst was filtered over a celite bed and the filtrate evaporated under reduced pressure. The residue was dried at 2 mm Hg pressure to obtain 5.0 g of the titled product as white foam. The obtained hydroxy compound material was used as such for the next reaction.

### Step 2: Synthesis of difluoro{[(2S,5R)-[7-oxo-2-{[(2S)-pyrrolidin-2-ylmethoxy]carbamoyl}-1,6-diaza-bicyclo[3.2.1]oct-6-yloxy]acetic acid ethyl ester

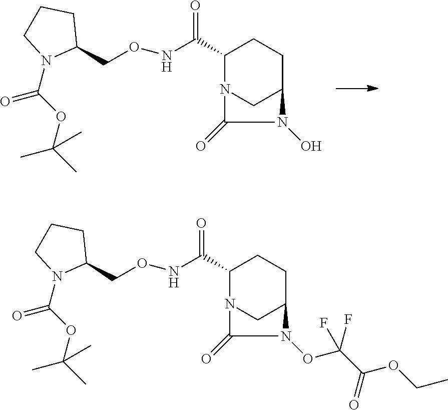 [SMILES: CC(C)(C)OC(=O)N1CCC[C@H]1CONC(=O)[C@@H]1CC[C@@H]2CN1C(=O)N2O ; CCOC(=O)C(F)(F)ON1C(=O)N2C[C@H]1CC[C@H]2C(=O)NOC[C@@H]1CCCN1C(=O)OC(C)(C)C]

A solution of hydroxy compound obtained in Step-1 (4.80 g, 0.0126 mol) N,N-dimethyl formamide (50 ml) was cooled to 0° C. and potassium carbonate (5.24 g, 0.038 mol) was added under stirring and inert atmosphere. Further, ethyl bromodifluoroacetate (6.01 g, 0.0296 mol) was added, and the suspension was stirred at room temperature for 4 hours. After completion of the reaction, the suspension was diluted with ethyl acetate (30 ml), filtered through a bed of celite and filtrate was washed with water (50 ml). The aqueous layer was again extracted with ethyl acetate (2×100 ml). The pooled organic layers were dried over sodium sulphate, solvent was removed under reduced pressure. The obtained residue was purified by flash column chromatography using ethyl acetate: hexane (Gradient elution 10%->20%->30%->50%). Evaporation of the solvent from the combined fractions gave 2.0 g of the titled product as white solid in 32% yield.

Analysis:

¹H-NMR: δ_{H} (CDCl₃, 400 MHz) 4.38 (qd, 2H), 4.12 (bs, 1H), 4.03 (d, 1H), 3.93 (s, 1H), 3.85 (m, 1H), 3.72 (m, 1H), 3.41-3.30 (m, 2H), 3.27 (d, 1H), 3.13 (d, 1H), 2.32 (dd, 1H), 2.13 (m, 1H), 2.03-1.92 (m, 2H) 1.87 (m, 2H), 1.68 (bs, 2H), 1.45 (s, 9H), 1.38 (t, 3H);

Mass: 505.4 (M−1) for Molecular Formula: C₂₁H₃₂F₂N₄O₈ and Molecular Weight: 506.5

### Step 3: Synthesis of difluoro{[(2S,5R)-[7-oxo-2-{[(2S)-pyrrolidin-2-ylmethoxy]carbamoyl}-1,6-diaza-bicyclo[3.2.1]oct-6-yloxy]acetic acid

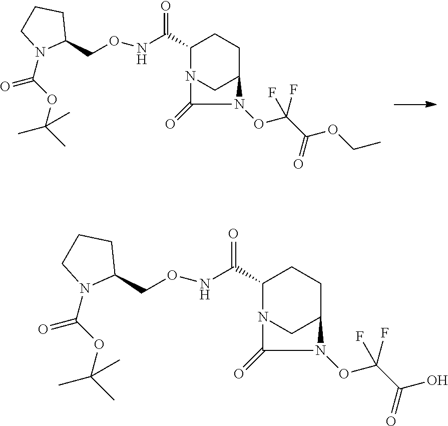 [SMILES: CCOC(=O)C(F)(F)ON1C(=O)N2C[C@H]1CC[C@H]2C(=O)NOC[C@@H]1CCCN1C(=O)OC(C)(C)C ; CC(C)(C)OC(=O)N1CCC[C@H]1CONC(=O)[C@@H]1CC[C@@H]2CN1C(=O)N2OC(F)(F)C(=O)O]

A solution of an ester compound obtained in step-2 (100 mg, 0.198 mmol) in tetrahydrofuran (1.6 ml) and water (0.4 ml) was cooled to −5° C. and lithium hydroxide monohydrate (10 mg, 0.213 mmol) was added under inert atmosphere. The solution was stirred at −3° C. to −5° C. until disappearance of the ester (about 3 hours). The pH of the obtained reaction mixture was adjusted to 3-4 with 1N HCl. The reaction mixture was extracted with dichloromethane (3×50 ml). The combined organic layer was dried over sodium sulphate and solvent was evaporated under reduced pressure and obtained residue was dried at 2 mm Hg to provide 62 g of the titled product as white foam in 66% yield.

Analysis:

¹H-NMR: δ_{H} (CDCl₃, 400 MHz) 4.12 (m, 1H), 4.01 (dd, 2H), 3.84 (m, 2H), 3.36 (m, 4H), 3.29 (d, 1H), 3.16 (m, 1H), 2.27 (dd, 1H), 2.14 (m, 1H), 1.99 (m, 2H), 1.88 (m, 2H), 1.45 (s, 9H);

Mass: 477.4 (M−1) for Molecular Formula: C₁₉H₂₈F₂N₄O₈ and Molecular Weight: 478.4.

### Step-4: Synthesis of difluoro{[(2S,5R)-[7-oxo-2-{[(2S)-pyrrolidin-2-ylmethoxy]carbamoyl}-1,6-diaza-bicyclo[3.2.1]oct-6-yloxy]acetic acid

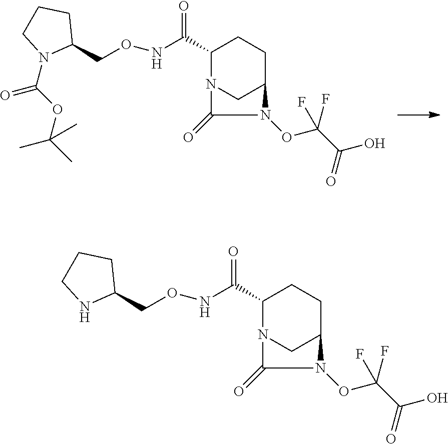 [SMILES: CC(C)(C)OC(=O)N1CCC[C@H]1CONC(=O)[C@@H]1CC[C@@H]2CN1C(=O)N2OC(F)(F)C(=O)O ; O=C(NOC[C@@H]1CCCN1)[C@@H]1CC[C@@H]2CN1C(=O)N2OC(F)(F)C(=O)O]

The product obtained in step 3 (80 mg, 0.167 mmol) was dissolved in dichloromethane (400 μl) under inert atmosphere. The obtained solution was cooled to −10° C. and to this was added pre-cooled trifluoroacetic acid (400 μl). The reaction mixture was stirred at −10° C. and monitored by ES-MS. After completion, the reaction mixture was diluted with hexane (4.0 ml) and hexane layer was decanted. The reaction mixture was further washed sequentially with hexane (1×4.0 ml), ether (2×4.0 ml), acetonitrile (2×2.0 ml) and ether (2×4.0 ml). The obtained residue was dried under reduced pressure to obtain 45 mg of the titled product as white solid in 72% yield.

Analysis:

¹H-NMR (DMSO-d₆, 400 MHz): δ 3.97 (m, 2H), 3.88 (dd, 1H), 3.76 (bs, 1H), 3.35 (m, 4H), 3.18 (bs, 2H), 3.09 (s, 1H), 2.02 (m, 2H), 1.92-1.86 (m, 2H), 1.77 (m, 1H), 1.59 (m, 1H);

Mass: 379.2 (M+1) for Molecular Formula: C₁₄H₂₀F₂N₄O₆ and Molecular Weight: 378.3

Examples 2 to 8 were prepared using the procedure described as in Example 1, wherein compound of Formula (II) with appropriate R′ is used as starting reagent in place of (2S,5R)-2-[(6-benzyloxy-7-oxo-1,6-diaza-bicyclo[3.2.1]octane-2-carbonyl)-methoxycarbamoyl]-(2S)-pyrrolidine-1-carboxylic acid tert-butyl ester.

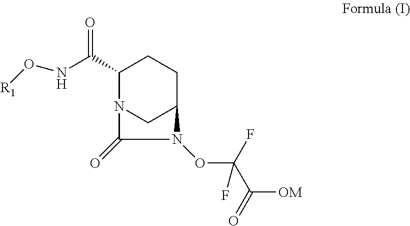

TABLE 1

| Ex- | | | | Mass |
|---|---|---|---|---|
| am- | | | | (as free acid) |
| ple | | | | Molecular |
| No. | Compound | R′ | ¹H NMR | Formula |
| 2. | 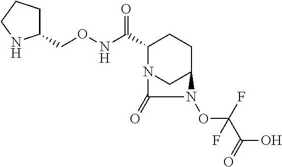 [SMILES: O=C(NOC[C@H]1CCCN1)[C@@H]1CC[C@@H]2CN1C(=O)N2OC(F)(F)C(=O)O] | 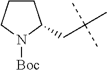 | ¹H NMR (400 MHz,): δ 11.76 (s, 1H), 8.82 (d, 2H, J = 120.4 Hz), 4.02-3.87 (m, 4H), 3.75 (brs, 2H), 3.18 (brs, 2H), 3.13 (s, 1H), 2.04-1.55 (m, 8H) | 379.3 (M + 1) C₁₄H₂₀N₄O₆F₂ |
| 3. | 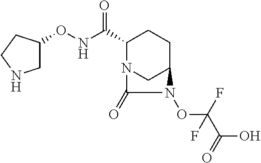 | 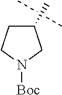 | ¹H NMR (400 MHz, DMSO-d₆) δ 8.89 (brs, 2H), 7.85 (s, 1H), 4.63 (s, 1H), 3.89-3.86 (m, 2H), 3.38- 3.26 (m, 4H, protons merged in water of DMSO), 3.06 (s, 2H), 2.30 (m, 1H), 2.13-1.92 (m, 4H), 1.76- 1.74 (m, 1H) | 365.1 (M + 1) C₁₃H₁₈F₂N₄O₆ |
| 4. | 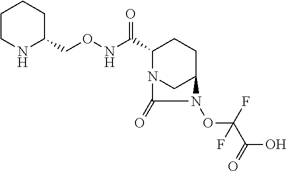 [SMILES: O=C(NOC[C@H]1CCCCN1)[C@@H]1CC[C@@H]2CN1C(=O)N2OC(F)(F)C(=O)O] | 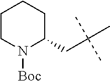 | ¹H NMR (400 MHz, CDCl₃): δ 11.9 (s, 1H), 8.5 (s, 2H), 3.3-3.9 (m, 7H), 3.14 (s, 3H), 2.84-2.87 (q, 2H), 2.05-2.07 (q, 2H), 1.73-1.83 (m, 5H), 1.37-1.57 (m, 4H). | 393.3 (M + 1) C₁₅H₂₂F₂N₄O₆ |
| 5. | 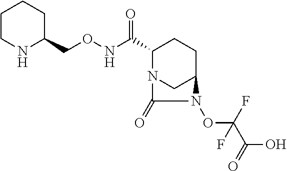 [SMILES: O=C(NOC[C@@H]1CCCCN1)[C@@H]1CC[C@@H]2CN1C(=O)N2OC(F)(F)C(=O)O] | 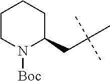 | ¹H NMR (400 MHz, CDCl₃): δ 11.9 (s, 1H), 8.5 (s, 2H), 3.3-3.9 (m, 7H), 3.14 (s, 3H), 2.84-2.87 (q, 2H), 2.05-2.07 (q, 2H), 1.73-1.83 (m, 5H), 1.37-1.57 (m, 4H). | 393.3 (M + 1), C₁₅H₂₂F₂N₄O₆ |
| 6. | 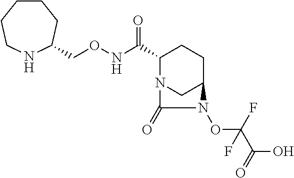 [SMILES: O=C(NOC[C@H]1CCCCCN1)[C@@H]1CC[C@@H]2CN1C(=O)N2OC(F)(F)C(=O)O] | 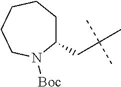 | ¹H NMR (DMSO•D₂O, 400 MHz): δ 3.90 (m, 2H), 3.44-3.23 (m, 5H), 3.10 (m, 2H), 2.04-1.90 (m, 3H), 1.76-1.68 (m, 5H), 1.51 (m, 3H), 1.07 (m, 1H) | 407.2 (M + 1), C₁₆H₂₄F₂N₄O₆ |
| 7. | 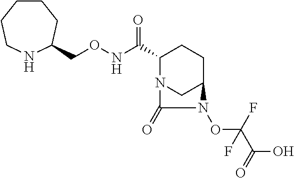 [SMILES: O=C(NOC[C@@H]1CCCCCN1)[C@@H]1CC[C@@H]2CN1C(=O)N2OC(F)(F)C(=O)O] | 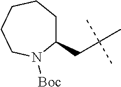 | ¹H NMR (DMSO•D₂O, 400 MHz): δ 3.96 (m, 3H), 3.45 (bs, 2H), 3.25- 3.22 (m, 1H) 3.14-3.03 (m, 3H), 2.09-2.06 (m, 2H), 1.96 (m, 2H), 1.80-1.71 (m, 6H), 1.521 (m, 2H). | 407.2 (M + 1), C₁₆H₂₄F₂N₄O₆ |
| 8. | 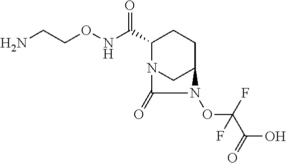 [SMILES: NCCONC(=O)[C@@H]1CC[C@@H]2CN1C(=O)N2OC(F)(F)C(=O)O] | 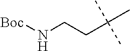 | ¹H NMR (DMSO, 400 MHz): δ 11.69 (br s, 1H), 7.94 (br s, 3H), 3.89-3.98 (m, 3H), 3.29-3.31 (m, 2H), 3.02-3.08 (m, 3H), 1.74-2.05 (m, 4H) | 339.1 (M + 1) C₁₁H₁₆F₂N₄O₆ |

### Biological Activity Data

The biological activity of representative compounds according to the invention against various bacterial strains was investigated. In a typical study, overnight grown bacterial cultures were diluted appropriately and inoculated on the agar media containing doubling dilutions of the test compounds. Observations for growth or no growth was performed after 16-20 hours of incubation at 35±2° C. in the ambient air. The overall procedure was performed as per Clinical and Laboratory Standards Institute (CLSI) recommendations, (Clinical and Laboratory Standards Institute (CLSI), Performance Standards for Antimicrobial Susceptibility Testing, 20^{th} Informational Supplement, M07-A9, Volume 32, No. 2, 2012). Molten Mueller Hinton Agar (MHA) (BD, USA) containing serial dilutions of each antibacterial agent were poured on to the plates and allowed to solidify. Appropriate suspensions from the freshly grown cultures were prepared in normal saline so that about 10⁴ CFU/spot of the organism was delivered on to the drug containing agar plates using automated multipoint inoculator (Mast, UK). The plates were incubated in Biochemical oxygen demand (BOD) incubator at 37° C. for 18 hours and then examined for growth. MICs were read as the lowest concentration of drug that completely inhibited bacterial growth.

The Table 2 depicts the antibacterial activity profile of compounds according to present invention against various multidrug resistant bacterial strains.

TABLE 2
Antibacterial activity of representatives of compound of Formula (I) ( expressed as MICs (mcg/ml)

| | | KP | | | | | | | | | | | K. pneumoniae | | | |
|---|---|---|---|---|---|---|---|---|---|---|---|---|---|---|---|---|
| | Com- | ATCC | E. coli | E. coli | E. coli | E. coli | E. coli | E. coli | E. coli | E. coli | E. coli | E. coli | H | H | H | H |
| Sr. | pounds | 700603 | 13351 | 13352 | 13353 | M 36 | M 50 | 7 MP | M 49 | M 138 | S 18 | H 483 | 521 | 522 | 523 | 525 |
| 1. | Example 1 | >32 | 2 | 2 | 2 | 2 | 2 | 4 | 8 | 2 | 2 | — | 4 | 2 | 2 | 2 |
| 2. | Example 2 | >32 | 1 | 1 | 0.5 | 0.5 | 1 | 2 | 2 | 1 | — | — | 1 | 1 | 1 | 1 |
| 3. | Example 3 | >32 | 4 | 4 | 4 | 4 | 4 | 8 | 8 | 4 | 16 | >32 | 4 | 4 | 4 | 4 |
| 4. | Example 4 | >32 | — | 1 | 1 | 1 | 2 | 2 | 4 | 2 | 2 | >32 | 2 | 1 | 1 | 1 |
| 5. | Example 5 | >32 | 8 | 4 | 4 | 4 | 4 | 8 | 8 | 4 | 4 | — | 4 | 4 | 4 | 4 |
| 6. | Example 6 | >32 | 2 | 2 | 1 | 1 | 2 | 4 | 8 | 2 | 2 | >32 | 2 | 2 | 2 | 1 |
| 7. | Example 7 | >32 | 2 | 2 | 4 | 4 | 2 | 4 | 2 | 4 | — | | 2 | 2 | 2 | 2 |
| 8. | Example 8 | >32 | 4 | 4 | 4 | 2 | 4 | 8 | 4 | 4 | 4 | >32 | 4 | 4 | 4 | 4 |

1. A compound of Formula (I): 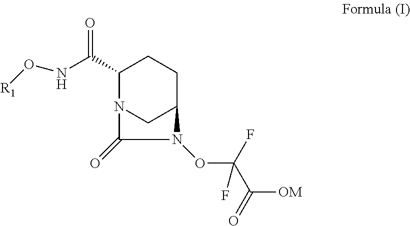
or a stereoisomer or a pharmaceutically acceptable derivative thereof;
wherein:
R₁ is selected from:
(a) hydrogen,
(b) C₁-C₆ alkyl optionally substituted with one or more substituents independently selected from CN, halogen, OR₂, NR₂R₃, COOR₂, CONR₂R₃, SR₂, cycloalkyl, heterocycloalkyl, aryl or heteroaryl,
(c) three to seven membered cycloalkyl optionally substituted with one or more substituents independently selected from CN, halogen, OR₂, NR₂R₃, COOR₂, CONR₂R₃ or SR₂,
(d) three to seven membered heterocycloalkyl optionally substituted with one or more substituents independently selected from CN, halogen, OR₂, NR₂R₃, COOR₂, CONR₂R₃ or SR₂,
(e) six to fourteen membered aryl optionally substituted with one or more substituents independently selected from CN, halogen, OR₂, NR₂R₃, COOR₂, CONR₂R₃, or SR₂, or
(f) five to fourteen membered heteroaryl optionally substituted with one or more substituents independently selected from CN, halogen, OR₂, NR₂R₃, COOR₂, CONR₂R₃ or SR₂;
R₂ and R₃ are each independently selected from:
(a) hydrogen,
(b) C₁-C₆ alkyl optionally substituted with one or more substituents independently selected from CN, halogen, OH, OCH₃, OC₂H₅, NH₂, NHCH₃, NHC₂H₅, COOH, CONH₂ or SR₂; and
M is hydrogen, cation or C₁-C₆ alkyl.

2. The compound according to claim 1, selected from:
Difluoro {[(2S,5R)-[7-oxo-2-{[(2S)-pyrrolidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2S)-pyrrolidin-2-ylmethoxy]carbamoyl}-1,6-diaza-bicyclo [3.2.1]oct-6-yloxy]acetic acid;
Difluoro {[(2S,5R)-[7-oxo-2-{[(2R)-pyrrolidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2R)-pyrrolidin-2-ylmethoxy]carbamoyl}-1,6-diaza-bicyclo [3.2.1]oct-6-yloxy]acetic acid;
Difluoro {[(2S,5R)-[7-oxo-2-{[(3S)-pyrrolidin-3-yloxy]carbamoyl}-1,6-diaza-bicyclo[3.2.1]oct-6-yloxy]acetic acid ethyl ester;
Difluoro {[(2S,5R)-[7-oxo-2-{[(3S)-pyrrolidin-3-yloxy]carbamoyl}-1,6-diaza-bicyclo[3.2.1]oct-6-yloxy]acetic acid;
Difluoro {[(2S,5R)-[7-oxo-2-{[(3R)-pyrrolidin-3-yloxy]carbamoyl}-1,6-diaza-bicyclo[3.2.1]oct-6-yloxy]acetic acid ethyl ester;
Difluoro {[(2S,5R)-[7-oxo-2-{[(3R)-pyrrolidin-3-yloxy]carbamoyl}-1,6-diaza-bicyclo[3.2.1]oct-6-yloxy]acetic acid;
Difluoro {[(2S,5R)-[7-oxo-2-{[(2R)-piperidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2R)-piperidin-2-ylmethoxy]carbamoyl}-1,6-diaza-bicyclo [3.2.1]oct-6-yloxy]acetic acid;
Difluoro {[(2S,5R)-[7-oxo-2-{[(2S)-piperidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2S)-piperidin-2-ylmethoxy]carbamoyl}-1,6-diaza-bicyclo [3.2.1]oct-6-yloxy]acetic acid;
Difluoro {[(2S,5R)-[7-oxo-2-{[(2R)-azepan-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2R)-azepan-2-ylmethoxy]carbamoyl}-1,6-diaza-bicyclo [3.2.1]oct-6-yloxy]acetic acid;
Difluoro {[(2S,5R)-[7-oxo-2-{[(2S)-azepan-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2S)-azepan-2-ylmethoxy]carbamoyl}-1,6-diaza-bicyclo [3.2.1]oct-6-yloxy]acetic acid;
({(2S,5R)-2-[(2-aminoethoxy)carbamoyl]-7-oxo-1,6-diazabicyclo[3.2.1]oct-6-yl}oxy) (difluoro)acetic acid ethyl ester;
({(2S,5R)-2-[(2-aminoethoxy)carbamoyl]-7-oxo-1,6-diazabicyclo[3.2.1]oct-6-yl}oxy) (difluoro)acetic acid;
Difluoro {[(2S,5R)-[7-oxo-2-{[(2R)-azetidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2R)-azetidin-2-ylmethoxy]carbamoyl}-1,6-diaza-bicyclo [3.2.1]oct-6-yloxy]acetic acid;
Difluoro {[(2S,5R)-[7-oxo-2-{[(2S)-azetidin-2-ylmethoxy]carbamoyl}-1,6-diaza-bicyclo [3.2.1]oct-6-yloxy]acetic acid ethyl ester;
Difluoro {[(2S,5R)-[7-oxo-2-{[(2S)-azetidin-2-ylmethoxy]carbamoyl}-1,6-diaza-bicyclo [3.2.1]oct-6-yloxy]acetic acid;
or a stereoisomer or a pharmaceutically acceptable derivative thereof.

3. The compound according to claim 1, selected from:
Sodium salt of difluoro {[(2S,5R)-[7-oxo-2-{[(2S)-pyrrolidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2R)-pyrrolidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(3S)-pyrrolidin-3-yloxy]carbamoyl}-1,6-diaza-bicyclo[3.2.1]oct-6-yloxy]acetic acid;
Sodium salt of difluoro {[(2S,5R)-[7-oxo-2-{[(3R)-pyrrolidin-3-yloxy]carbamoyl}-1,6-diaza-bicyclo[3.2.1]oct-6-yloxy]acetic acid;
Sodium salt of difluoro {[(2S,5R)-[7-oxo-2-{[(2R)-piperidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-piperidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2R)-azepan-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-azepan-2-ylmethoxy]carbamoyl}-1,6-diaza-bicyclo[3.2.1]oct-6-yloxy]acetic acid;
Sodium salt of ({(2S,5R)-2-[(2-aminoethoxy)carbamoyl]-7-oxo-1,6-diazabicyclo[3.2.1]oct-6-yl}oxy)(difluoro)acetic acid;
Sodium salt of difluoro {[(2S,5R)-[7-oxo-2-{[(2R)-azetidin-2-ylmethoxy]carbamoyl}-1,6-diaza-bicyclo[3.2.1]oct-6-yloxy]acetic acid;
Sodium salt of difluoro {[(2S,5R)-[7-oxo-2-{[(2S)-azetidin-2-ylmethoxy]carbamoyl}-1,6-diaza-bicyclo[3.2.1]oct-6-yloxy]acetic acid;
or a stereoisomer thereof.

4. A pharmaceutical composition comprising a compound according to any one of claims 1 to 3.

5. The pharmaceutical composition according to claim 4, further comprising at least one antibacterial agent or a pharmaceutically acceptable derivative thereof.

6. The pharmaceutical composition according to claim 5, wherein the antibacterial agent is selected from a group consisting of aminoglycosides, ansamycins, penems, carbapenems, carbacephems, cephalosporins, cephamycins, lincosamides, lipopeptides, macrolides, monobactams, nitrofurans, penicillins, polypeptides, quinolones, sulfonamides, tetracyclines, or oxazolidinone antibacterial agents.

7. The pharmaceutical composition according to claim 5, wherein the antibacterial agent is a beta-lactam antibacterial agent.

8. The pharmaceutical composition according to claim 5, wherein the antibacterial agent is selected from a group consisting of cephalothin, cephaloridine, cefaclor, cefadroxil, cefamandole, cefazolin, cephalexin, cephradine, ceftizoxime, cefoxitin, cephacetrile, cefotiam, cefotaxime, cefsulodin, cefoperazone, ceftizoxime, cefmenoxime, cefmetazole, cephaloglycin, cefonicid, cefodizime, cefpirome, ceftazidime, ceftriaxone, cefpiramide, cefbuperazone, cefozopran, cefepime, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil or cefditoren pivoxil, cefuroxime, cefuroxime axetil, loracarbacef, ceftaroline, ceftolozane, latamoxef, piperacillin, imipenem, doripenem, and meropenem.

9. The compound according to any one of claims 1 to 3 for use in a medicament useful in the treatment of a bacterial infection.

10. The pharmaceutical composition according to any one of claims 4 to 8 for use as a medicament useful in the treatment of a bacterial infection.
